(19) United States
(12) Patent Application Publication
(10) Pub. No.: US 2003/0096015 A1
(43) Pub. Date: May 22, 2003

(54) Cytogenic/nucleogenic healing

(76) Inventor: Sharwan Kumar Kakar, Bayville, NY (US)

Correspondence Address: SHARWAN KUMAR KAKAR, 37 JUNE AVENUE, BAYVILLE, NY, 11709, US

(21) Appl. No.: 10/246,403

(22) Filed: Sep. 18, 2002

Related U.S. Application Data
(63) Continuation-in-part of application No. 09/649,034, filed on Aug. 25, 2000.
Application No. 09/649,034 is a continuation of application No. 08/990,993, filed on Dec. 15, 1997, now abandoned.

(51) Int. Cl. A61K 35/78; A61K 35/20; A61K 31/70; A61K 31/20

(52) U.S. Cl. 424/535; 424/750; 424/757; 424/777; 514/23; 514/558

(57) ABSTRACT

The present invention provides a method as well as a composition for treating disorders of gastrointestinal tract and more specifically, for treating colon cancer. Method of treatment comprises the steps of administering to the human with colon cancer a composition comprising proteins-rich, lipids-rich and carbohydrates-rich food types. The method involves the use of forward flux of tissues growth factors and finds use in tissues cultures, animals and plants.

### CROSS REFERENCE TO RELATED APPLICATION

The present application is a continuation-in-part of U.S. application Ser. No. 09/649,034, filed Aug. 25, 2000, and entitled “Cytogenic/Nucleogenic Healing”, the entire contents and disclosure of which are hereby specifically incorporated by reference. The said U.S. application Ser. No. 09/649,034 is a continuation of U.S. application Ser. No. 08/990,993, filed Dec. 15, 1997 and entitled “Cytogenic/Nucleogenic Healing”, the entire contents and the disclosure of which are hereby specifically incorporated by reference. The said application Ser. No. 08/990,993 has “improved version CIP”, mailed May 12, 1998, entitled “Cytogenic/Nucleogenic Healing” with no official notification and with no serial number granted, the entire contents and disclosure are hereby incorporated by reference. The present amended application also contain some part of contents derived from “the enclosed CIP specification”, filed Aug. 9, 1999, paper no. 5, with no Serial Number granted, the entire contents and disclosure of which are hereby specifically incorporated by reference.

### STATEMENT REGARDING FEDERALLY SPONSORED RESEARCH OR DEVELOPMENT

Not Applicable

### REFERENCE TO A MICROFICHE APPENDIX

Not Applicable

### BACKGROUND OF THE INVENTION

1. Field of Invention

The present invention pertains to compositions and methods of treating disorders of gastrointestinal tract and more specifically, the present invention relates to a composition and methods for treating colon cancer.

2. Description of the Prior Art

Pathologic disorders of lower digestive tract comprise a group of conditions characterized clinically by an abnormal epithelial cell proliferation. Before the development of a colonic neoplasm, colonic epithelial cells undergo a series of multistage process including atrophy, hyperplasia, dysplasia and neoplasia.

The term neoplasm is reserved for the formation of benign and, more particularly, for malignant tumors. Tumor is a more general term signifying a swelling or lump but there are many lumps that are not neoplasm such as abscesses. A benign tumor is slow growing and is contained. In contrast, malignant tumor is invasive and tends to metastasize.

Cancer has been linked to multi-causative factors including chemical, environmental, enzymatic, genetic, hormonal, immunologic, metabolic and viral. German pathologist Rudolf Virchow (in 1847) suggested that irritation could set up inflammatory processes in epithelium causing it to become malignant. Later Berrenblum (in 1947) suggested that tumor formation is a multistage process. Viral origin of various types of cancer was demonstrated by many pathologists including Ellermann and Bang (in 1908), Rouse (in 1911), Shope (in 1932), Vitar (in 1936) and Gross (in 1950s). Stewart and Eddy discovered the polyoma virus capable of producing multiple kinds of cancer in animals. Green (in 1954) advanced the theory of carcinogenesis based upon immunologic differences, Baker G., Oncology, GOOD HOUSE KEEPING FAMILY HEALTH & MEDICAL GUIDE, The Hearst Corporation, 162-164, 1979.

An important step in the fight against cancer is a recognition by a pathologist of the earliest development of the malignant process. The disease is curable when the malignant change is still cytologic rather than histologic in origin. This state of affairs is characterized as carcinoma-in-situ. The condition may be reversible but in many cases, the in-situ state develops into an invasive one, Boyd W. and Sheldon H.; Introduction to the Study of Disease, 8th Ed. Philadelphia, Lea and Febiger, 239, 257, 1980. The prevalent state of art deals with three major forms of treatment including surgery, radiation therapy and chemotherapy. The principal treatment for gastrointestinal cancers is usually surgical, preceded by radiation therapy or chemotherapy or both.

U.S. Pat. No. 5,198,250 entitled “FOOD AND PHARMACEUTICAL COMPOSITIONS CONTAINING SHORT CHAIN MONOUNSATURATED FATTY ACIDS AND METHODS OF USING” discloses methods and compositions to treat atherosclerotic lesions and U.S. Pat. No. 5,214,062 entitled: “METHODS AND COMPOSITION FOR TREATING IMMUNE DISORDERS, INFLAMMATION AND CHRONIC INFECTIONS” discloses methods to treat chronic infections. See also U.S. Pat. No. 5,118,673 and U.S. Pat. No. 5,703,060 entitled: “USES OF ALOE PRODUCTS”.

### SUMMARY OF THE INVENTION

Accordingly, it is the object of the present invention to provide a composition and methods that prior art fails to teach.

Another object is to provide a composition comprised of six food ingredients which will bring about gradual improvements including bowel movement control, regression of cancerous colonic polyps and enhanced neuro-musculo-vasculoskeletal functions in patients with colonic neoplasm.

An additional object is to provide a composition for treating colon cancer in a human comprising the steps of administering to the human a composition sufficient in amount to induce replacement of modified tissues associated with colon cancer, by generating forward flux of healthy functional tissues.

A further object is to provide a composition and methods for treating more common disorders of the gastrointestinal system.

The detailed description of the preferred variants of the invention and the specific examples appearing herein after may be referred to for a more complete and comprehensive understanding of invention and are not intended as being limited and/or to the spirit and the scope of the appended claims.

### BRIEF DESCRIPTION OF THE DRAWINGS

FIG. 1 is a representation of “Three Component Combinatorial Model” illustrating interaction between percent concentrations of carbohydrates, lipids and proteins-nucleic acid components in living systems and their relationship in growth process and pathogenesis.

### DETAILED DESCRIPTION OF THE INVENTION

Cytogenic/Nucleogenic Healing © is the technical name given by the assignee of the, instant invention to the technique of treating and/or curing a wide variety of pathologic disorders of mammals. This technique involves the use of cells as well as cellular constituents derived from animal and plant kingdoms to promote regulated healthy tissue growth in living systems.

The detailed description will hereafter be given of the present invention being a method for treating colon cancer. In an embodiment, a method for treating a colon cancer in a human is provided comprising the steps of administering to the human a composition comprising of about 50% proteins-rich food, 30% lipids-rich food and 20% carbohydrates-rich food.

In an embodiment, the composition of proteins-rich food comprises about 25% soybeans, 15% milk and 10% vegetables.

In an embodiment, the composition of lipids-rich food comprises about 30% butter fats.

In an embodiment, the composition of carbohydrates-rich food comprises about 15% whole wheat and 5% fruits.

In an embodiment, a method of treating colon cancer in a human is provided comprising the steps of administering to the human a composition comprising of about 25% soybeans, 15% milk, 10% vegetables, 30% butter fats, 15% whole wheat, and 5% fruits.

In an embodiment, a method of treating disorders of gastrointestinal tract in a human is provided comprising the steps of administering to the human a composition comprising of about 25% soybeans, 15% milk, 10% vegetables, 30% butter fats, 15% whole wheat and 5% fruits.

### Definition of Terms

The term “cell” as used herein means cells of animals and plants. The cells that are found in animals include adipose cells, chondrocyte, epithelial cells, erythrocytes, fibroblasts, immune cells, myoblasts, pancreatic cells, neuroblasts, osteocytes and stem cells.

The term “food constituents” as used herein relates to the classification of biological constituents into three categories: proteins-rich food having high peptide contents as found in tissues of animals and plants, polysaccharides-rich food having high glycoside contents as found in barley, corn, oats, potatoes, rice and wheat and lipids-rich food having high methylene contents as found in oils, fats and waxes.

The term “polysaccharides” as used herein means compounds with many saccharides units. Polysaccharides and complex carbohydrates are interchangeable terms. Glucose, and sugar are simple saccharides, whereas starch and glycogen are complex polysaccharides. Associated with connective tissues of animals and plants are groups of unusual polysaccharides like mucopolysaccharides which includes hyaluronic acid, chondroitin sulfate, and heparin. Pure carbohydrates are herein identified as oxidants and are designated as inflammatory agents.

The term “proteins” as used herein means food with relatively high peptide contents like animal proteins and plant proteins. Animal proteins include beef, lamb, pork, poultry, seafood, eggs and milk. Plant proteins include vegetables, nuts and seeds with high DNA contents. Proteins with alkyl chains are designated as anti-inflammatory agents.

The term “lipids” as used herein means oils, fats and waxes that are found in tissues of animals and plants. Vegetables oils like olive oil and mustard oil are liquid at room temperature and are identified as partial reductants. Saturated fats and waxes are solid at room temperature. Fats are herein identified as reductants and are designated as anti-inflammatory agents.

The “term” plant as used herein includes algae, grasses, bushes, herbs, vines and trees. The body of a typical plant includes roots, root system, stems, leaves, buds, flowers, fruits and seeds.

The “seed” as used herein means an immature embryo containing nutritive reserves with protective coat. It has all the elements for growth and is a good source of food for animals, organisms and plants.

The “term” vegetables used herein means species of plants that include algae, alfalfa, artichoke, asparagus, beets, bamboos, broccoli, brussell sprouts, celery, chicory, cabbage, cauliflower, garlic, green beans, carrot, chick peas, chives, cucumber, egg plant, flax, ginger, gourd, luffa, onion, horse radish, mustard, okra, olive, papaya, peas, potatoes, peppers, radish, spinach, turnip, tomato, squash, pumpkin, zucchini, anise, basil, coriander, fennel, pepper grass and parsley. These species provide chloroplasts and proteins.

The “term” fruits as used herein includes apple, apricot, avocado, banana, blueberry, cantaloupe, cherry, cranberry, currant, gooseberry, grapes, grape fruit, guava, litchi, lime, lemon, mango, orange, peach, pineapple, plum, quince, raspberry and strawberry with high contents of terpenes, saccharide, polysaccharide, and citric acid cycle products.

The term “atrophy” as used herein means decrease in the size and/or number of cells of a particular tissue or an organ after it has achieved its normal size.

The term “hyperplasia” as used herein means an increase in the number of cells of a body part. It merges with the part of neoplasia.

The term “dysplasia” as used herein means an abnormality in the maintenance or development of a tissue.

The term “electrolyte balance” as used herein means the delicate balance of major electrolytes with air and water. Major electrolytes include sodium, potassium, calcium, magnesium, iron, manganese and zinc in form of acetate, citrate, fluoride, chloride, iodide, in form of ionophors, chlorophyll, cytochromes, protoporphyrins and proteinates

The term “extracellular matrix” or ECM as used herein means the basement membranes and its components including mucopolysaccharides, mucoproteins, collagens and hyaluronates. The mucosa as used herein relates to ECM that lies beneath the surface of epithelial cells. This layer is produced by chondrocytes and fibroblasts.

### DEVELOPMENT OF CYTOGENIC/NUCLEOGENIC MODEL

The objective of this study was to find a zone where optimum yield of healthy tissue growth takes place. Human body for its survival and healthy development needs several components including lipids, proteins, genetic components, polysaccharides, minerals, vitamins, oxygen and water. In this study the concentrations of vitamins, minerals, oxygen and water were kept constant, the response Y in term of status of health

Y=f{L}{P:N}{S} (1)

where {L}, {P: N} and {S} are respective concentrations of lipids, proteins-nucleic acid components and polysaccharides that enter into the cell or body.

### Experimental

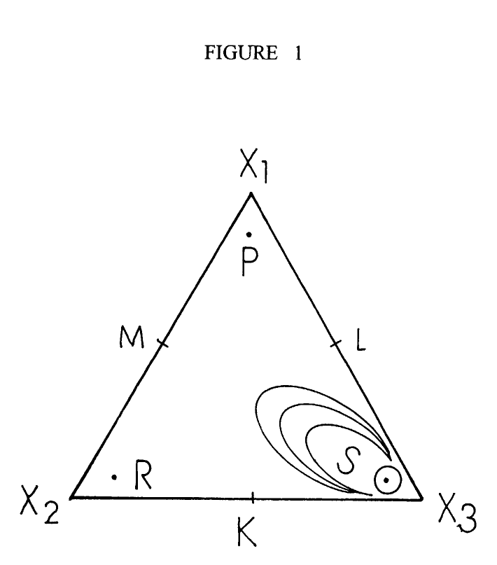

Three Component Combinatorial Model was developed by using Experimental Design Technique to establish a zone where healthy response in term of growth, development and survival could be achieved. FIG. 1 shows the graphic representation of response versus percent composition of food constituents. Constituents X₁, X₂, and X₃ denote respective concentrations of carbohydrates, saturated lipids and proteins:nucleic acid components. At respective points K, L, and M the respective concentrations of X₁, X₂ and X₃ is zero.

### Materials and Methods

The source of plant proteins-rich food was fresh vegetables, legumes, including soybeans, kidney beans, black eye beans, lima beans, chick peas, mung and peas. The source of animal proteins-rich food was beef, lamb, pork, veal, poultry, eggs, seafood and milk. The source of lipids-rich food was butter fats, cream and coconut. This was also the source of oil soluble vitamins. The source of saccharides and polysaccharides-rich food was sugar, fruits, whole wheat, barley, corn, millets, oats, rice, potatoes and seaweed. Fruits and grains were also the source of water soluble vitamins and minerals. The source of refined carbohydrates was sugar and unbleached flour. The source of casein was cheese and source of refined oil was corn oil. Legumes, grains, seaweeds and fresh vegetables were obtained from Asian grocery stores. Cheese, corn oil, unbleached flour, potatoes, vitamin-mineral pills were obtained from local super market. Fresh milk, cream and butter were obtained from local farms. In these farms animals were fed on natural feed including corn, corn stalks, grass, hay, oats, vegetables, fruits, and seaweeds and were raised in a clean fresh-air environment. All ingredients used in this study were either boiled, cooked, steamed or fried and were consumed within twelve hours. Unconsumed meals were stored in refrigerator not more than twelve hours.

The following compositions with food constituents in various ratios were selected for evaluation in term of pathogenesis, human survival and healthy human development.

A. Diet containing 80% unbleached flour, 10% corn oil and 10% casein and one daily vitamin-mineral pill ˆ

B. Diet containing 10% whole wheat, 80% butter fats and 10% soybeans*

C. Diet containing 15% whole wheat, 5% fruits⁺, 30% butter fats, 25% soybean, 15% milk and 10% vegetables!

D. Diet containing 5% whole wheat, 5% fruits, 10% butter fats, 55% soybeans, 15% milk, and 10% vegetables NOTE: %denotes dry % wt/wt, A FDA daily requirement, 37 vegetables, + 22 fruits, * 7 legumes

A subsequent experiment was conducted using human subjects over a period of two years. Ten individuals who took part in this study were young, healthy, active vegetarian males in their twenties as volunteers with no previous medical history of any disease. Appearance of lesions in form of tumor, ulceration, indigestion and repeated infections was graded as an unhealthy state. Disappearance of symptoms was graded as survival or a healthy state

### Results

Individuals who consumed dietary intake A acquired diseases of ECM including allergies, common cold, chronic infections, diabetes, high blood pressure, leukemia, and age related autoimmune syndrome over a period of one year. Individuals who consumed dietary intake of composition B developed indigestion after one meal. No further study was conducted with this specific composition. Individuals who developed aforementioned disorders with composition A, were then maintained on a regimen of dietary intake of composition C and composition D. These individuals were cleared off their symptoms over a period ranging from six months to a year and developed resistance to repeated bacterial, fungal and viral infections. FIG. 1 shows the plot of response versus percent compositions of food constituents. Point P represents a region where unhealthy response was obtained when the ratio of food constituents was maintained 8:1:1. This region was designated as a zone of pathogenesis. Point R represents a region which was found to be devoid of biological activity when the ratio of food constituents was maintained at 1:8:1. Point S represents a region where healthy response in term of survival and human development was obtained when the ratios of food constituents was maintained at 2:3:5 and 1:1:8 respectively. These ratios which directly correspond to composition C and composition D respectively, were found to be helpful in reversing aging and many age related degenerative disorders of ECM. Zone S was labeled as a region of survival and healthy human development. It was also designated as a region of regulated cell growth.

Similar correlation were found with other proteins-rich food including meat, poultry, eggs sea food, lipids-rich food including cream, lard and coconuts, and carbohydrates-rich food including corn, potatoes, rice and seaweeds. The following model was designed to establish the relationship between various biological constituents.

$$\begin{matrix}Gene Expression \\ or \\ Growth Factor\end{matrix} = ∇ \frac{\{ L \} \{ P : N \}}{\{ S \}} {C}_{k} \qquad ( 2 )$$

where Cₖ is “Kakar cell constant” of any living system and represents the inherited cellular machinery of a fertilized egg or a target cell type. The symbol delta <∇>denotes Operator Stimulus that acts upon living cell or body to stimulate growth through its entire life span. Thus stimulatory factors including gravity, temperature, pressure, static electricity, fresh air, message, mental discipline, yoga, special exercises, gardening and walking become integral part of cytogenic/nucleogenic healing techniques.

### Experimental

### Generation of Epithelial Tissue Growth Factor in vitro

The objective of this study was to investigate the potential of compositions in optimized zone to generate epithelial tissue growth factors. The growth of epithelial cells is largely dependent upon the surrounding connective tissues was originally proposed in 1903 and even earlier, Renaut, des se' ances de la soc, de biol, 55: 1620, 1903. It was pointed that a fibrillar component of connective tissue was of significant importance for the growth of nearby epithelial cells. Fibrillar components of ECM have a vital role to play in the control of cell shape, cytoskeletal bodies and biosynthesis in embryonic and mesenchymal tissue formation, Gresh and Cachpole, American Journal of Anatomy, 85: 457, 1949; Hay, Journal of Cellular Biology, 91: 45, 1945; Hay, journal of Cellular Biology, 91: 2055, 1981; and Langer and Vicanti, Science, 260: 920, 1991.

In this investigation, healthy epithelial tissues were obtained from hand pads of the female patient with skin cancer on the front side of her right foot and were kept in a buffer solution for further use. After washing with buffer solution, the isolated epithelial cells were implanted on a layer of algae previously treated with a thin film of ECM in a petri dish containing 0.5% solution of aforementioned compositions at ambient temperature. Cells were occasionally stimulated by exposing to air and sunlight. Culture medium was changed every 72 h. After three weeks, epithelial tissues about 4.0 cm. in diameter were harvested. The harvested cells were then implanted on the cancerous lesion of the same patient to investigate if it could lead to tissue growth. Initially, cancerous lesions failed to respond to outer in-situ application but after the institution of optimized compositions progressive regression in abnormal epithelial cell proliferation and increased expression of epidermal keratinocytes were noticed. Complete closure of cancerous lesion occurred after one year with the appearance of young healthy skin.

For comparative study, experiments were conducted with 0.5% solutions of sucrose and butter fats respectively. Rapid cell replication was observed in form of sticky clumps with no distinct differentiation into tissue formation, with sucrose solution. These sticky clumps were later invaded by fungi and bacteria after 48h, whereas no cellular replication was observed in a medium containing butter fats. It was further observed that medium with butter fats when streaked with culture containing mixed colonies of multiple of infections (MOI) including candida, streptococcus viridans, staphylococcus aureus, streptococcus pneumonia, porphyromonas gingivitis, salmonella typhosa, mixed multiple influenza viruses and HIV-viruses, showed no bacterial and viral growth for several months, where as bacterial activity and viral replication at exponential rate were observed with sucrose solution after five hours under similar experimental conditions. Likewise, studies were conducted with oils, animal fats and waxes.

These studies revealed that fats and virgin oils had high degree of resistance to proteolytic activities caused by multiple of infections (MOI) as compared to refined oils. Fats were also found to be equally effective agents in generating stem cells.

It became evident that compositions in optimized region had the potential to generate epithelial growth factors that could lead to healthy tissue formation. In a similar manner, nerve tissues with extensive dendrites formation were harvested using isolated nerve cells as seeding elements on various supporting structures. Additional growth factors including adipose tissues growth factors, chondrocytes growth factors, connective tissues growth factors, dendrites tissues growth factors, erythrocytes growth factors, immune cells growth factors, fibroblasts growth factors, leucocytes growth factors, melanocytes growth factors, myoblasts growth factors, leucocytes growth factors, mesenchyme tissues growth factors, neuroblasts growth factors, osteocytes growth factors, plasma cells growth factors, pancreas tissues growth factors, pericytes growth factors, mitochondrial growth factors, stem cells growth factors and reticuloendothelial tissues growth factors were generated in vitro and in vivo by using the following scheme. These growth factors were termed as Kakar forward flux of cytogenic/nucleogenic growth factors.

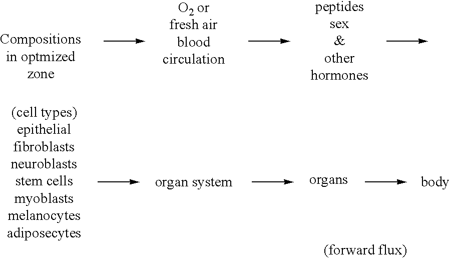

The results of some of the experiments are shown in Table 1.

TABLE 1
Compositions With Potential FOR Generating Various Tissues Growth
Factors AND Their Resistance To Proteolysis By MOI AT Ambient
Temperature (24° C.)

| | Comparative Degree | Relative | Potential for |
|---|---|---|---|
| Composition | of Resistance (h.) | Resistance | Tissue Growth |
| 5% Sucrose | 5.0 | 1.0 | sticky lump |
| Butter Fats | 8.6 × 10³ | 5.65 | NTG |
| Composition A | 12.0 | 1.55 | + |
| Composition B | 4.3 × 10³ | 5.2 | NTG |
| Composition C | 5.06 × 10² | 3.87 | +++++ |
| Composition D | 1.44 × 10² | 3.09 | +++++ |

NOTE:
NTG referred as no tissue growth, + referred as degree of healthy tissue growth

Results in Table 1 indicate that composition C and composition D have the potential to generate healthy tissues as compared to composition A. Comparative evaluation of composition C and D further reveals that composition C had high degree of resistance to proteolysis by MOI as compared to composition D. Composition C was selected for treating patients who were at greater risk in the development of colonic neoplasm whereas composition D was designed for patients who had excessive amount of adipose tissues in their body.

Common disorders of gastrointestinal tract include acidic stomach, peptic ulcer, ulcerative colitis, Crohn's disease, Zollinger-Ellison's syndrome and dysfunctional stomach. Cancer that are commonly encountered in gastrointestinal tract include lips, tongue, salivary glands, esophagus, stomach and colon. Predisposing factors that lead to colon cancer include ulcerative colitis and papillomatous conditions of intestine.

The objective of this study was to investigate if cytogenic/nucleogenic healing technology could be substituted for chemotherapy because the current treatment and chemoprevention have not significantly improved the poor survival rate of patients with adenocarcinoma of colon and also for patients who had undergone polypectomy

The selected optimized-composition was studied for detailed therapeutic evaluation with patients who were at greater risk for the development of colonic neoplasm. To assess the effectiveness of the treatment, a patient with a recent case history of colonic cancerous polyps was recruited. Biopsy specimens were taken from lower section of the colon at the time of onset of the trial, then at intervals of one year and three years respectively. These specimens were clinically examined, tested, data were recorded and compared with the normal control. Histologic tests included epithelial cell proliferation, keratinocyte expression, DNA contents and colonoscopy.

### Method of Administration

The cytogenic/nucleogenic properties of the composition permits it to be used in/on inflamed tissues and organ in-situ of mammals. It may be administered daily in form of soup. The best way to administer is in form of meals at least three or four times a day at a rate of about 1.0-2.0 g/kg/day or more. This composition may be compressed into dosage units like pills, tablets or candies. Topical application may be in form of powder, processed gel, supporting flexible materials, cream, lotion, ointment and coated film. For topical application, the concentration of formulation can contain up to 20% of composition.

### EXAMPLE

A 70 year old vegetarian Asian lady developed colitis of lower digestive tract i.e. colon. CAT scan revealed the existence of multiple tumors of liver. She had a history of colonic adenoma and had antibodies against mucopolysaccharides. She was at greater risk in the development of colonic adenocarcinoma. Histological evaluation of biopsy specimens of lower section of colon confirmed the presence of abnormal tissue proliferation. Administration of optimized formulation over a period of one year resulted in disappearance of adenomas and sloughing off abnormal mucosa by new layers of epithelium with normal cellular differentiation and maturation. Follow up on the patient was continued up to three years. The response to the treatment was quite effective with gradual improvement in mucosal functions. The average pre- and post treatment results of the patients are shown as follows:

| Tests | Onset | 1 Year | 3 Years |
|---|---|---|---|
| Epithelial Cell expression | +++\hat{ } | + | NC |
| Keratinocyte expression | 0 | +++ | NC |
| Cell shape | distorted | NC | NC |
| Nuclei size | large & HC | NC | NC |
| DNA contents | transformed | NC | NC |
| Colonoscopy | 4.95 | 0.5 | NC |

NOTE:
\hat{ }denotes slightly below abnormal, HC denotes hyperchromatic, NC denotes normal control

Patient showed significantly reduced level of abnormal epithelial cell proliferation and increased level of keratinocyte expression after one year which is usually observed in normal functioning colon. The absence of transformed DNA contents was noticed approaching normal control classification after one year. This treatment restored the normal flora which stimulated healthy epithelial tissue growth and eliminated flora associated with MOI. No adverse effects were noticed with this treatment.

This nutritional approach was also used for patients who had undergone surgery. Two patients with adenocarcinoma of colon who had undergone polypectomy showed no recurrence of disease when maintained on this regimen. These patients are still alive, well and showing great deal of exuberance about life in their advanced years with complete absence of discomfort in their gastrointestinal tract after a decade.

### Method of Preparation

To administer the optimized-composition in form of soup for treating patients with colon cancer, the following steps were taken:

1. Soybeans and whole wheat were cooked in pan with one liter of water for 25 minutes at medium heat.

2. To this finely chopped vegetables, butter fats and fresh milk were added. This mixture was boiled for another 10 minutes and then allowed to cool.

3. The cooked mixture was stirred with required amount of fresh fruits in a blender and made just salty enough to suit patient's taste prior to ingestion.

4. A fresh cup of prepared soup was administered in the morning to the patient with colon cancer. The rest of the soup was administered three or four times a day at regular intervals of four hours. During this period, electrolyte balance was regularly monitored under a physician care. Mild physical activities were instituted at this stage.

After adjusting to the above formulation for four weeks, the same composition was administered to the patient in solid or semisolid form.

To Prepare the composition in form of powder or candy bar the following steps were taken:

Ingredients including soybeans and butter fats were added in a pan and were stirred at medium heat till they turned slightly brown. After cooling, dehydrated powdered milk, dehydrated powdered fruits and dehydrated powdered vegetables were added to the mixture. Stirring was continued till the contents reached room temperature. After cooling, mixture was ground to powder and was converted into candy bar of specific sizes by extrusion process. The powdered material was given as a snake in form of shake in between meals.

Later soybeans were substituted with other protein-rich food including chick peas, mung, legumes, lentils and animal proteins, whole wheat was substituted with seaweed, corn, potatoes, and rice and butter fats were substituted with cream and coconut milk to enhance patient's palatability towards various food.

Administration of the optimized composition caused gradual amelioration of symptoms over a period of six months. During this period normal functioning of bowel movement was noticed. Complete neuromuscular control over bowel was noticed after one year.

It should be understood that each of the food ingredient described above, or two or more in various proportions differing from aforementioned compositions were also useful in curing other types of disorders differing from the types quoted above. While the novel features of this invention have been described and are pointed out in annexed claims, it will be understood that various omissions, modifications, substitutions and changes in the forms and details of the above invention and in its operation can be made without departing in any way from the spirit and scope of the present invention. It is therefore intended that such change and modifications be covered by the appended claims.

I claim:

44. A method for treating colon cancer in a human, comprising the steps of: administering to the human a composition comprising about 25% soybeans, 15% milk, 10% vegetables, 30% butter fats, 15% whole wheat and 5% fruits.

45. A method for treating colon cancer in a human, comprising the steps of: administering to the human a composition comprising about 50% proteins-rich food, 30% lipids-rich food and 20% carbohydrates-rich food.
